(19) United States
(12) Patent Application Publication
(10) Pub. No.: US 2006/0074349 A1
(43) Pub. Date: Apr. 6, 2006

(54) Blood sampler

(75) Inventor: Tzu-Sheng Fan, Miao Li Hsien (TW)

Correspondence Address: BACON & THOMAS, PLLC, 625 SLATERS LANE, FOURTH FLOOR, ALEXANDRIA, VA, 22314, US

(73) Assignee: TAIJECT MEDICAL DEVICE CO., LTD., Miaoli (TW)

(21) Appl. No.: 11/224,284

(22) Filed: Sep. 13, 2005

(30) Foreign Application Priority Data
Oct. 1, 2004 (TW) 93215654

(51) Int. Cl. A61B 5/00

(52) U.S. Cl. 600/576

(57) ABSTRACT

A blood sampler is disclosed to include a needle holder for holding a needle, and a barrel, which has a front neck for holding the needle holder and the attached needle in the operative position, a longitudinal sliding slot for guiding movement of the needle holder and the attached needle from the operative position to the received position inside the barrel after the application of the blood sampler, a turning slot for guiding the needle holder from the front neck into the longitudinal sliding slot, and a retainer provided at the bottom side of the longitudinal sliding slot to secure the needle holder to the received position.

### BACKGROUND OF THE INVENTION

1. Field of the Invention

The present invention relates generally to a blood sampler and more particularly, to a simple structure of blood sampler that is easy to assembly and convenient to use.

2. Description of the Related Art

Various blood samplers are well known and intensively used in hospitals and examination centers for collecting blood from patients for examination. These blood samplers commonly have the needle holder designed to be receivable with the attached needle inside the barrel after the application to prevent contamination. According to conventional designs, the needle holder is mounted in the barrel from the rear side. This back loading design has the needle holder fit the diameter of the receiving chamber of the barrel, resulting in an inconvenience in installation.

### SUMMARY OF THE INVENTION

The present invention has been accomplished under the circumstances in view. It is the main object of the present invention to provide a blood sampler, which has a simple structure and is easy to assemble. It is another object of the present invention to provide a blood sampler, which can easily be operated to receive the needle inside the barrel after the application.

To achieve these and other objects of the present invention, the blood sampler comprises a barrel for receiving a blood vial, and a needle holder mounted in the barrel for holding a needle. The barrel comprises a hollow cylindrical body, which defines a receiving chamber and an insertion hole at the bottom side of the receiving chamber, a holder passage portion formed to the front side of the hollow cylindrical body, a neck perpendicularly forwardly extended from the holder passage portion and defining an axial hole and a side opening in communication with the axial hole, a longitudinal sliding slot longitudinally cut through the peripheral wall of the hollow cylindrical body and extending to the holder passage portion, a turning slot in communication between the side opening of the neck and the longitudinal sliding slot, and a guide means formed integral with the inside wall of the hollow cylindrical body and extending along one side of the longitudinal sliding slot. The needle holder comprises a needle connector fitted into the axial hole of the neck and defining therein a hole for holding a needle, a finger strip for gripping with the fingers to move the needle connector and the attached needle into the inside of the barrel after the application of the blood sampler, a connecting bar connected between the needle connector and the finger strip and set in the side opening of the neck, and guide means for coupling to the guide means of the barrel to guide movement of the needle holder along the longitudinal sliding slot.

### BRIEF DESCRIPTION OF THE DRAWINGS

FIG. 1 is an exploded view of a blood sampler according to the present invention.

FIG. 2 is an elevational view of a needle holder for blood sampler according to the present invention.

FIG. 3 is a perspective assembly view of the present invention, showing the blood sampler ready for receiving a blood vial.

FIG. 4 is a perspective view of the present invention, showing the needle holder of the blood sampler turned to the right in vertical alignment with the longitudinal sliding slot of the barrel.

FIG. 5 is similar to FIG. 4 but showing the needle holder moved to a midway toward the bottom side of the longitudinal sliding slot of the barrel.

FIG. 6 is similar to FIG. 5 but showing the needle holder moved to the received position, the needle received inside the barrel.

FIG. 7 is a sectional elevation of the present invention, showing the closed status of the sealing cap at the bottom side of the hollow cylindrical body of the barrel.

FIG. 8 is a perspective view of a blood sampler according to a second embodiment of the present invention.

FIG. 9 is similar to FIG. 8 but showing the needle holder turned to the right in vertical alignment with the longitudinal sliding slot of the barrel.

FIG. 10 is a sectional view of the second embodiment of the present invention, showing the needle holder and the needle received inside the barrel.

### DETAILED DESCRIPTION OF THE INVENTION

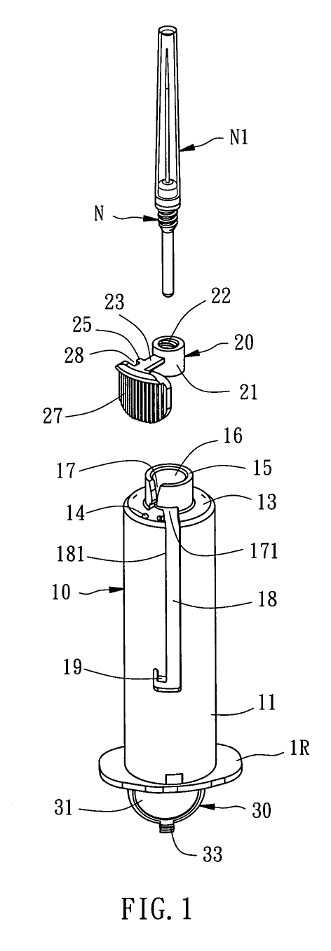

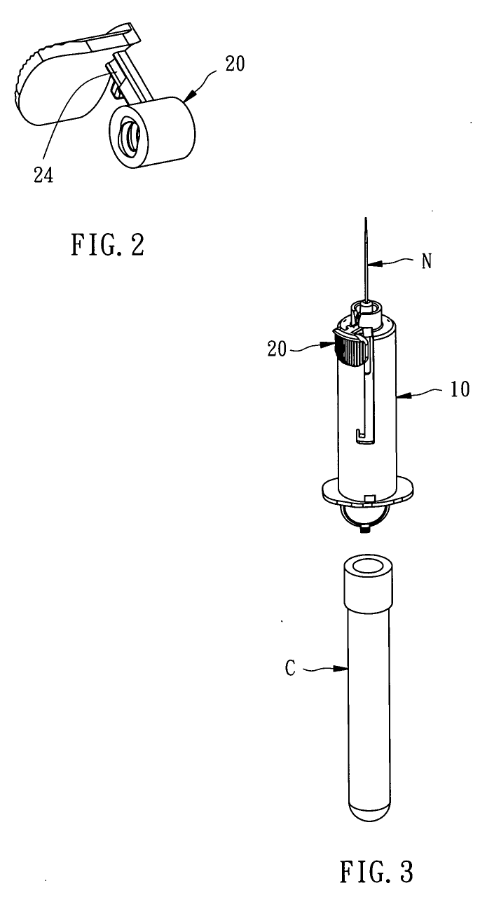

Referring to FIGS. 1˜7, a blood sampler in accordance with the first embodiment of the present invention is shown to be used with a needle N capped with a needle protector N1 for taking blood from the body of a patient for further examination. The blood sampler comprises a barrel 10, a needle holder 20, and a sealing cap 30.

The barrel 10 comprises a hollow cylindrical body 11 defining a receiving chamber 12 and an insertion hole 111 at the bottom side of the receiving chamber 12, a holder passage portion 13 formed to the front side of the hollow cylindrical body 11, a locating portion 14 of raised emboss provided at the front side of the holder passage portion 13, a neck 15 perpendicularly forwardly extended from the holder passage portion 13 at the center and defining therein an axial hole 16 and a side opening 17 in communication with the axial hole 16, a longitudinal sliding slot 18 longitudinally cut through the peripheral wall of the hollow cylindrical body 11 and extending to the holder passage portion 13, a guide means, for example, longitudinal rib 181 formed integral with the inside wall of the hollow cylindrical body 11 and extending along one side of the longitudinal sliding slot 18 for guiding movement of the needle holder 20 along the longitudinal sliding slot 18 stably, a retainer 19 of inner curved or Y-shaped shape formed in the hollow cylindrical body 11 at the bottom end of the longitudinal sliding slot 18, a turning slot 171 formed in the holder passage portion 13 in communication between the side opening 17 and the top end of the longitudinal sliding slot 18, and a finger flange 1R extended around the periphery of the insertion hole 111 at the rear side of the hollow cylindrical body 11.

The needle holder 20 comprises a needle connector 21 fitted into the axial hole 16 of the neck 15 and defining therein a needle insertion hole 22 of screw hole for holding the needle N, a finger strip 27 for gripping with the fingers to move the needle connector 21 and the attached needle N into the inside of the barrel 10, a connecting bar 23 connected between the needle connector 21 and the finger strip 27 and set in the side opening 17 of the neck 15, a guide means, for example, guide groove 28 for coupling to the longitudinal rib 181 to guide movement of the needle holder 20 along the longitudinal sliding slot 18 stably, a locating hole 24 disposed at the bottom side of the connecting bar 23 for receiving the locating portion 14 to hold the needle holder 20 firmly in the neck 15, and a retaining portion 25 protruding from one lateral side of the connecting bar 23.

The sealing cap 30 comprises a flexible cap body 31 hinged to the bottom side of the hollow cylindrical body 11 of the barrel 10 for closing the insertion hole 111 and a retaining portion 33 for fastening to a part of the hollow cylindrical body 11 to lock the flexible cap body 31 after the flexible cap body 31 has been closed on the bottom side of the hollow cylindrical body 11 to seal the insertion hole 111.

Referring to FIGS. 1˜3, after installation of the needle N in the needle connector 21 of the needle holder 20 and removal of the needle protector N1 from the needle N, the needle connector 21 and connecting bar 23 of the needle holder 20 are respectively engaged into the axial hole 16 and side opening 17 of the neck 15 of the barrel 10 for force the locating hole 24 into engagement with the locating portion 14. Thereafter, the blood vial C can be inserted into the barrel 10 and connected to the needle N to collect blood from the body of the patient.

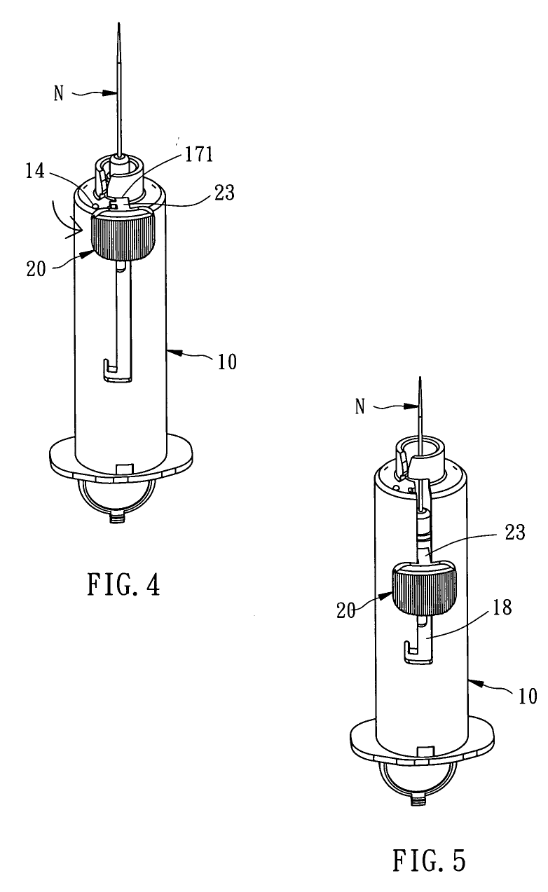

Referring to FIGS. 4 and 5, after collection of blood from the body of the patient, the blood vial C is removed from the blood sampler, and the finger strip 27 of the needle holder 20 is turned to the right to disengage the locating hole 24 from the locating portion 14, and then the finger strip 27 is pulled downwards to move the guide groove 28 along the longitudinal rib 181 and to lower the needle holder 20 along the longitudinal sliding slot 18.

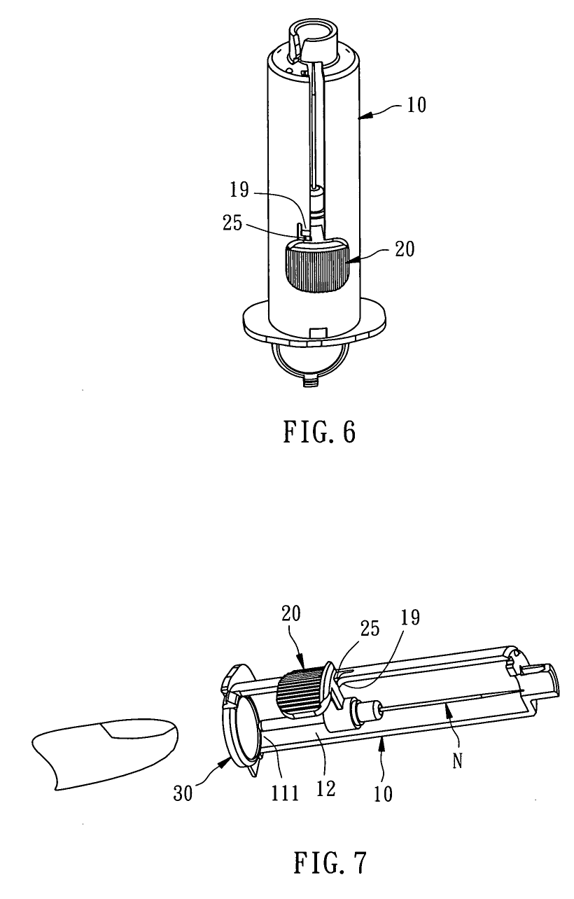

Referring to FIGS. 6 and 7, after the needle holder 20 has been moved to the bottom side of the longitudinal sliding slot 18, the retaining portion 25 of the needle holder 20 is forced into engagement with the retainer 19 of the barrel 10, prohibiting forward displacement of the needle holder 20 relative to the barrel 10, and then the sealing cap 30 is sealed to the insertion hole 111 at the rear side of the hollow cylindrical body 11 of the barrel 10 to prevent insertion of the finger into the inside of the barrel 10 accidentally.

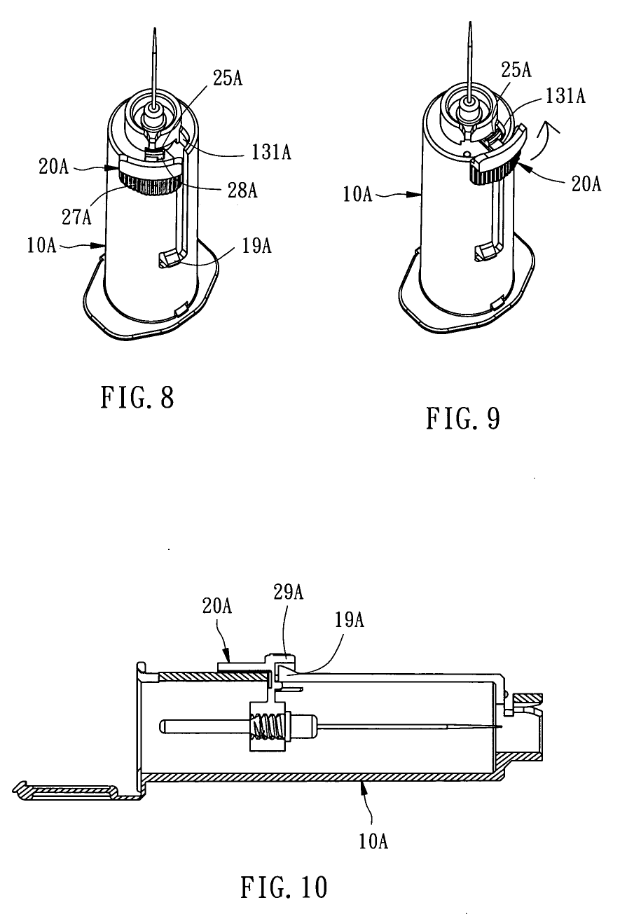

FIGS. 8˜10 show an alternate form of the present invention. Similar to the aforesaid first embodiment of the present invention, the blood sampler according to this second embodiment is comprised of a barrel 10A and a needle holder 20A. The barrel 10A and the needle holder 20A are substantially similar to like parts in the aforesaid first embodiment with the exceptions described hereinafter. The retainer 19A of the barrel 10A is an outer protrusion shape provided at the outside wall of the barrel 10A. The barrel 10A further has a stop wall 131A adapted to limit the turning angle of the finger strip27A when the user shifting the needle holder 20A from the side opening of the neck of the barrel 10A into the longitudinal sliding slot of the barrel 10A. The retaining portion 25A and the guide groove 28A of the needle holder 20A are disposed at the other side of the connecting bar when compared to the aforesaid first embodiment. Further, the finger strip 27A is engageable with the retainer 19A of the barrel 10A, having a shielding wall 29A, which shields the retainer 19A of the barrel 10A after engagement of the finger strip 27A with the retainer 19A of the barrel 10A.

The assembly procedure and operation of the aforesaid second embodiment are substantially same as the aforesaid first embodiment.

As indicated above, the invention has the following advantages:

1. The barrel and the needle holder have a simple structure and are easy to assemble.

2. The needle holder can easily smoothly be moved relative to the barrel between the extended position and the received position and firmly held in received position after the application.

What is claimed is:

1. A blood sampler comprising:
a barrel for receiving a blood vial, said barrel having a hollow cylindrical body, said hollow cylindrical body defining a receiving chamber and an insertion hole at a bottom side of said receiving chamber, a holder passage portion formed to a front side of said hollow cylindrical body, a neck perpendicularly forwardly extended from said holder passage portion, said neck defining an axial hole and a side opening in communication with said axial hole, a longitudinal sliding slot longitudinally cut through the peripheral wall of said hollow cylindrical body and extending to said holder passage portion, a turning slot in communication between said side opening of said neck and said longitudinal sliding slot, and a guide means formed integral with an inside wall of said hollow cylindrical body and extending along one side of said longitudinal sliding slot.
a needle holder mounted in said barrel for holding a needle, said needle holder having a needle connector fitted into the axial hole of said neck, said needle connector defining therein a hole for holding a needle, a finger strip for gripping with the fingers to move said needle connector into the inside of said barrel, a connecting bar connected between said needle connector and said finger strip and set in the side opening of said neck, and guide means for coupling to said guide means of said barrel to guide movement of said needle holder along said longitudinal sliding slot.

2. The blood sampler as claimed in claim 1, wherein said barrel comprises a locating portion provided at a front side of said holder passage portion; said needle holder comprises a locating hole for engagement with said locating portion of said barrel.

3. The blood sampler method as claimed in claim 1, wherein the guide means of said needle holder is a guide groove coupled to the guide means of said barrel.

4. The blood sampler as claimed in claim 1, wherein said barrel further comprises a retainer formed in said hollow cylindrical body at a bottom side of said longitudinal sliding slot; said needle holder comprises a retaining portion for engagement with the retainer of said barrel after receiving of said needle holder and the attached needle on the inside of said barrel.

5. The blood sampler as claimed in claim 4, wherein said guide means of said needle holder and said retainer of said barrel are disposed at the same side in relation to said longitudinal sliding slot of said barrel.

6. The blood sampler as claimed in claim 4, wherein said guide means of said needle holder and said retainer of said barrel are disposed at the different sides in relation to said longitudinal sliding slot of said barrel.

7. The blood sampler as claimed in claim 6, wherein said barrel further comprises a stop wall disposed at one side of said turning slot and adapted to guide said needle holder into said longitudinal sliding slot upon turning of said finger strip by the user.

8. The blood sampler as claimed in claim 4, wherein said retainer of said barrel has the shape of inner curved, Y-shaped or outer protrusion.

9. The blood sampler as claimed in claim 4, wherein said finger strip of said needle holder comprises a shielding wall adapted to shield said retainer of said barrel after engagement of said retaining portion of said needle holder with said retainer of said barrel.

10. The blood sampler as claimed in claim 1, further comprising a sealing cap hinged to said hollow cylindrical body of said barrel and adapted to close the insertion hole of said hollow cylindrical body of said barrel.
